(19) United States
(12) Patent Application Publication
(10) Pub. No.: US 2004/0110957 A1
(43) Pub. Date: Jun. 10, 2004

(54) Process for the synthesis of an antiviral compound

(75) Inventors: Joerg Wilken, Hanover (DE); Frank Nerenz, Hanover (DE); Andreas Kanschik-Conradsen, Garbsen (DE)

Correspondence Address: SYNNESTVEDT & LECHNER, LLP, 2600 ARAMARK TOWER, 1101 MARKET STREET, PHILADELPHIA, PA, 191072950

(73) Assignee: Honeywell International, Inc., Morristown, NJ (US)

(21) Appl. No.: 10/660,837

(22) Filed: Sep. 12, 2003

Related U.S. Application Data
(60) Provisional application No. 60/431,112, filed on Dec. 5, 2002.

(51) Int. Cl. C 07D 4 5/02

(52) U.S. Cl. 546/282.1; 562/402

(57) ABSTRACT

In a process comprising synthesizing pyranes including [R—(R*,R*)]-N-[3-[1-[5,6-dihydro-4-hydroxy-2-oxo-6-(2-phenylethyl)-6-propyl-2H-pyran-3-yl]propyl]phenyl]-5-(trifluoromethyl)-2-pyridinesulfonamide the present invention comprises the improvements comprising: (a) providing a racemic mixture of 3-hydroxy-3-(2-phenylethyl)-hexanoate ethyl acetate by reacting said 1-phenyl-hexan-3-one with ethylbromoacetate under Reformatsky conditions; and (b) separating (R)-3-hydroxy-3-(2-phenylethyl)-hexanoic acid in enantiomeric excess by saponification and reverse resolution of the racemate of step (a) to produce a resolved product. In addition, the present invention comprises a reverse resolution process for separating an enantiomer from a mixture of enantiomers.

### REFERENCE TO RELATED APPLICATIONS

This application is based on an claims the priority of U.S. Provisional Application 60/431,112 filed Dec. 5, 2002.

### FIELD OF THE INVENTION

The present invention relates to improved processes for the synthesis and/or separation of optically active hydroxy acids. More particularly, the present invention relates to synthesis of (R) 3-hydroxy-3-(2-phenylethyl)-hexanoic acid (hereinafter “the (R) hydroxy acid”), and to improved methods of synthesizing the antiviral compound [R—(R*,R*)]-N-[3-[1-[5,6-dihydro-4-hydroxy-2-oxo-6-(2-phenylethyl)-6-propyl-2H-pyran-3-yl]propyl]phenyl]-5-(trifluoromethyl)-2-pyridinesulfonamide and related compounds of this class of compounds.

### BACKGROUND OF THE INVENTION

Antiviral activity is exhibited by some members of the 4-hydroxy-2-oxo-pyrane family of compounds and their derivatives (hereafter, “the antiviral compounds”). For example, [R—(R*,R*)]-N-[3-[1-[5,6-dihydro-4-hydroxy-2-oxo-6(2phenylethyl)-6-propyl-2H-pyran-3yl]propyl]phenyl]-5-(trifluoromethyl)-2-pyridinesulfonamide, has been identified as a protease inhibitor useful in managing Human Immunodeficiency Virus (HIV) infections in humans. The synthesis of the antiviral compound has been described in published patent applications WO 99/12919 (hereinafter, “the '919 application) and WO 00/55150, each of which is incorporated herein by reference. The antiviral compound synthesis described in the '919 application is shown in Scheme I, below.

As shown in Scheme I, the antiviral compound is an optically active compound. Of the possible isomers which can exist, only the one identified in Scheme 1 is known to exhibit antiviral activity. The activity of the antiviral compound is known to be inhibited by the presence of the other isomeric forms of the compound. Thus the pharmacological activity of the antiviral compound is dependant upon providing the compound in high optical purity.

It is known to synthesize the antiviral compound in a highly optically pure form by utilizing as a starting material the “R” enantiomer of 3-hydroxy-3-(2-phenylethyl)-hexanoic acid (herein referred to sometimes for convenience as the “R-acid”), as shown in Scheme I. Two routes for the synthesis of the antiviral compound from the R-acid are identified in Scheme I, labeled “Pathway A” and “Pathway “B”, and will be described in more detail below.

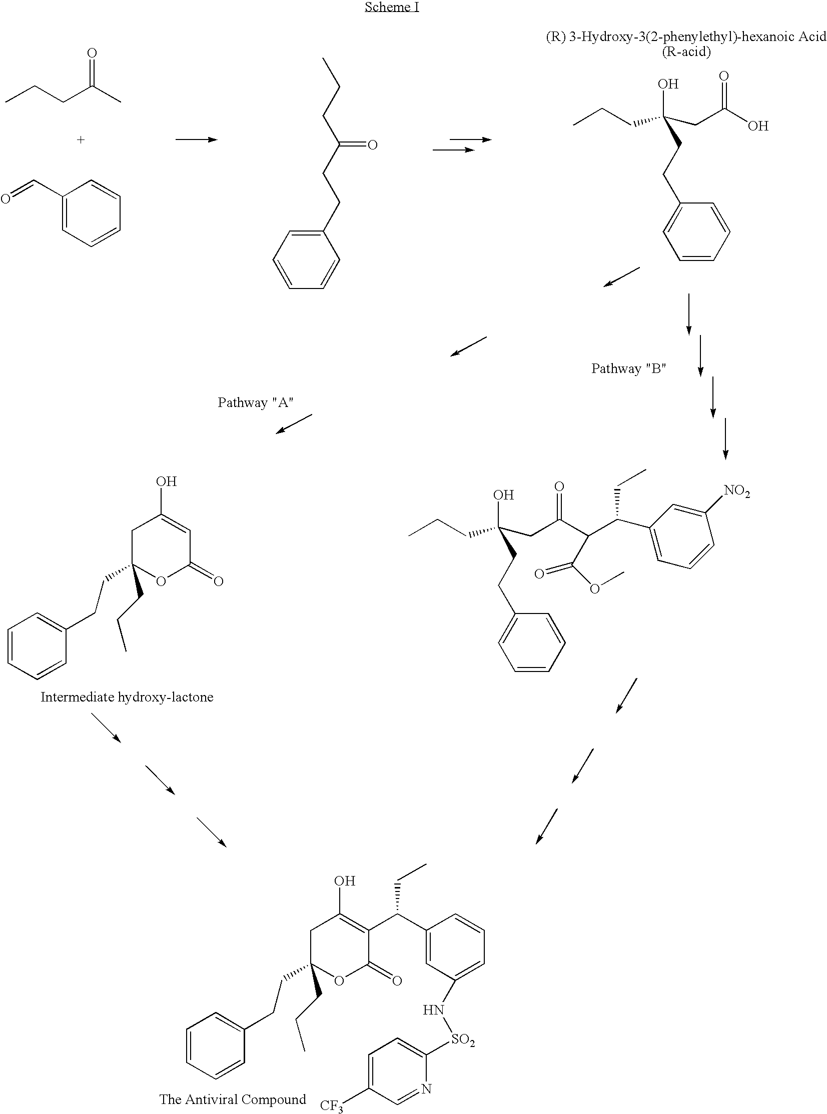

It is known in the art to prepare the hydroxy acid using an Aldol condensation reaction in which 1-phenylethyl-hexan-3-one is condensed with ethylacetate in the presence of lithium diisopropyl amide or butyl lithium. The hydroxy acid ester is thereby produced as a racemate and is liberated as the free acid upon acidification of the reaction mixture. Accordingly, it is known to resolve the racemic mixture of acids by crystallizing the (R) enantiomer from the racemic mixture using (1R)(2S)-norephedrine (hereinafter, (L)(−)norephedrine) and recovering it by filtration for use in the synthesis of the antiviral compound.

In “Pathway A” of the art-recognized synthesis, the R-acid is converted to (R)-ethyl-5-hydroxy-3-oxo-5-phenyethyl-octanoate (hereinafter, “the keto-ester”). The keto-ester is cyclized, yielding chiral intermediate 5,6-dihydro-α-pyrone (designated “intermediate hydroxy-lactone” in Scheme I). The intermediate hydroxy-lactone is further reacted with m-nitropropiophenone in the presence of titanium tetrachloride and pyridine yielding both enantiomers of [3α(R),6(R)]-5,6-dihydro-4-hydroxy-3-[1-(3-nitrophenyl)-propenyl]-6-[1-(2-phenylethyl)]-6-propyl-2H-pyran-2-one (hereinafter, “the nitro-propenyl compound”). The nitropropenyl compound is stereospecifically reduced to the corresponding [3α(R),6(R)]-5,6-dihydro-4-hydroxy-3-[1-(3-nitrophenyl)-propyl]-6-[1-(2-phenylethyl)]-6-propyl-2H-pyran-2-one (hereinafter, “the nitro-propyl compound”), followed by reduction of the 3-nitrophenyl functional group to 3-aminophenyl (hereinafter, “the amino-propyl compound”) in separate hydrogenation/reduction reactions. The antiviral compound is yielded by contacting the amino-propyl compound with 5-(trifluoromethyl)-2-pyridinesulfonyl chloride in the presence of pyridine and acidifying the mixture. The details of the synthesis are described in the aforementioned WO 99/12919.

In “Pathway B”, an uncyclized intermediate, (3R),(7R)-4-carbomethoxy-7-hydroxy-3-(3-nitrophenyl)-7-(2-phenylethyl)-decan-5-one (hereinafter, “the acylic ketone”), is prepared from the hydroxy acid in a multi-step reaction sequence as described in detail in the above-described published international application. The acylic ketone is then cyclized to provide directly the nitropropyl compound described above, which is subjected to the further reactions described above to yield the antiviral compound.

It will be appreciated that other synthetic schemes for the preparation of the antiviral compound from (R) 3-hydroxy-3-(2-phenylethyl)-hexanoic acid are possible, and may also be improved by the present process, as described and claimed hereinafter for synthesis of the R-acid and its resolution.

In view of these prior art processes for synthesizing the antiviral compound, it will be appreciated that the preparation of an antiviral agent of high activity is dependant upon preparation and separation techniques which provide a high yield of the critical intermediate (R) 3-hydroxy-3-(2-phenylethyl)-hexanoic acid in high optical purity.

It is known to prepare racemic 3-hydroxy-3-(2-phenylethyl)-hexanoic acid from the achiral ketone, 1-phenyl-hexan-3-one, via an Aldol condensation reaction with ethyl acetate. The aldol condensation provides a best known yield of up to about 95% conversion of the ketone to the hydroxy acid, that is, a racemic mixture of both enantiomers, thus, a yield of the hydroxy acid (R) enantiomer of less than about 48% based on the ketone starting material. To maximize yield from the Aldol condensation, temperatures in the range of between about −70° C. and about −58° C. must be maintained in the reaction mixture. In this temperature range, reaction rate is slow, but conversion of product back to starting materials via retro-Aldol reaction is suppressed. The retro-Aldol reaction becomes increasingly facile as the reaction temperature is increased, lowering overall reaction yield. In addition, the Aldol condensation reaction is induced by a powerful base, for example, lithium diisopropylamide and butyl lithium.

Applicants have come to recognize that the requirement for an agressive reagent to drive the reaction mitigates against running the reaction on a scale appropriate for the preparation of commercial quantities of the compound. In addition, applicants have come to appreciate that the reaction conditions described above for the Aldol condensation present engineering problems when the reaction is scaled to a size appropriate for the synthesis of commercial quantities of materials. Additionally, problems in heat transfer are usually encountered when attempting to maintain temperatures far below ambient with consistency throughout a large volume of a reaction mixture, especially in a reaction utilizing an agressive reagent which tends to liberate a large quantity of heat locally in the reaction mixture during the reaction.

The known process for separation of the R-acid from the acid racemic mixture, for example, a racemate produced by the Aldol condensation described above (also called resolution of the racemate), involves selective precipitation of the desired enantiomer from a solution of the racemate. Thus, high optical purity R-acid may be separated from a solution of the racemate by treating the solution with a resolving agent that forms a sparingly soluble complex with only the R-acid (but not with its enantiomer). In this manner, the desired enantiomer is preferentially precipitated from the solution. An example of such a resolving agent is L-(−)norephedrine, which forms a neutralization product with (R) 3-hydroxy-3-(2-phenylethyl)-hexanoic acid that precipitates out of an acetonitrile solution of the racemic acid. According to prior art processes, generally, several recrystallization steps must be carried out on the recovered precipitate to provide the level of purity of R-acid required for use in synthesis of pharmaceutical products.

This prior scheme has been applied to the resolution of R-acid from a racemate produced by the above-described Aldol condensation and the results reported by Fors et al. in A Convergent, Scalable Synthesis of HIV Protease Inhibitor PNU-140690, Journal of Organic Chemistry, vol 63 (1998) pp. 7348-56, which is incorporated herein by reference. The reported best chemical purity of the isolated material was about 98 mole % and the optical purity was about 98 enantiomeric excess. That is, with respect to the optical purity of the hydroxy acid isolated, 99 mole % was the R enantiomer and 1 mole % was the S enantiomer. With regard to yield, the total amount of the R-acid isolated was 54 mole % of the total R-acid produced in the condensation reaction. Thus, the prior art resolution process provided the R-acid in less than 27 mole % yield based on the starting 1-phenyl-hexan-3-one out of a theoretically possible 50 mole % yield.

Applicants have come to appreciate a need for processes for the preparation and separation of the R-acid in relatively high yield, and particularly yields substantially superior to those achieved by the prior art, based on the starting ketone, 1-phenyl-hexan-3-one, and the provision of processes for producing the R-acid which are more readily scalable to a size commensurate with the production of commercial quantities of the antiviral compound and structurally related 4-hydroxy-2-oxo-pyrane compounds which demonstrate antiviral activity.

### SUMMARY OF THE INVENTION

Applicants have discovered synthetic processes which tend to overcome some or all of the above-noted disadvantages of the prior art as well as others which are not mentioned above. One aspect of the present invention includes a process for producing racemic 3-hydroxy-3-(2-phenylethyl)-hexanoic acid (the hydroxy-acid) at relatively rapid reaction rates, and preferably in yields that are high compared to the prior art. Another aspect of the present invention is the provision of an improved process for resolving a hydroxy-acid racemate. These process aspects can be utilized independently, but preferably in combination, to provide improved synthesis of the antiviral compound, and can also be used in connection with achieving improvement of the synthesis of other optically active compounds.

One preferred form of the present invention involves a process for synthesizing 3-hydroxy-3-(2-phenylethyl)-hexanoic acid by reacting 1-phenyl-hexan-3-one with 2-bromo-ethylacetate, preferably under conditions which do not require any substantial cooling of the reaction mixture, and which preferably rely on reactants, reagents and catalyst systems that are stable and safe to store and/or use under ambient conditions. In the preferred embodiments, the reaction proceeds effectively under Reformatsky conditions, preferably at a temperature of from about 60° C. and about 100° C.

Another aspect of the present invention is the provision of a “reverse” resolution process (reverse resolution) for separating and preferrably substantially isolating, a first enantiomer (preferably of a chiral hydroxy acid, preferably in relatively high yield, and preferably in relatively high optical purity) from a mixture of the first enantiomer (which is desired to be obtained in a relatively optically pure form) and a second enantiomer, the presence of which is unwanted in the final product. In preferred embodiments, the process comprises:

a) providing a first solution comprising: the first enantiomer in acid form; the second enantiomer in acid form; and a polar, aprotic solvent;

b) contacting said first solution with a first resolving agent under conditions effective to precipitate from said first solution at least a portion of said second enantiomer (preferably at least about 20 percent, and even more preferably at least about 70 percent of said second enantiomer contained in said first solution), thereby forming a supernatant solution, while having at least about 90 percent (and even more preferably at least about 98 percent) of said first enantiomer remain in the supernatant solution;

c) transferring a substantial portion, preferably at least about 75 percent and even more preferably at least about 90 percent, of the first enantiomer contained in the supernatant solution into a second solution comprising said first enantiomer in acid form and a polar, aprotic solvent which is substantially free of said first resolving agent; and

d) contacting said second solution with a second resolving agent under conditions effective to precipitate at least a portion of said first enantiomer, (preferably at least about 90 percent, and even more preferably at least about 98 percent) contained in said second solution, said precipitate not containing a substantial amount of, and preferably being substantially free of, said second enantiomer.

Unless otherwise specifically indicated, the percentages identified herein are weight percentages. In preferred embodiments, the transferring step c) comprises: (1) separating the supernatant solution from the precipitate; (2) replacing at least a portion the polar, aprotic solvent in the separated supernatant solution (preferably at least about 50 percent, and even more preferably at least about 80 percent) with an aprotic, non-polar solvent to produce a substantially non-aqueous, non-polar composition comprising said first enantiomer and said first resolving agent; (3) contacting said non-aqueous, non-polar composition with a sufficient amount of an aqueous acid under conditions sufficient to cause at least a substantial portion of said first resolving agent to migrate from said non-aqueous, non-polar composition to said aqueous acid phase and to convert substantially all of said first enantiomer to an acid form; (4) separating the non-aqueous, non-polar composition containing said first enantiomer in acid form from said aqueous acid phase; and (5) replacing at least a portion the aprotic, non-polar, solvent in the separated non-aqueous, non-polar composition (preferably at least about 70 percent, and even more preferably at least about 90 percent) with a polar, aprotic solvent to produce said second solution comprising a polar, aprotic solvent and said first enantiomer.

Selection of the resolving agents is an important aspect of certain embodiments of the present invention. The first resolving agent is preferably characterized by the ability to form with the second enantiomer a precipitate that is at least only sparingly soluble in the polar, aprotic solvent and which does not form such a precipitate with the first enantiomer. Thus, the resolving agent in preferred embodiments is an important element in the formation of the first precipitate. While not wanting to be bound by theory, it is thought that in preferred embodiments the first precipitate contains the second enantiomer and the first resolving agent in the form of a complex. Likewise, the second resolving agent is preferably characterized by the ability to form a precipitate with the first enantiomer that is at least only sparingly soluble in the polar, aprotic solvent and does not form such a precipitate with the second enantiomer. Again, not wanting to be bound by theory, it is thought that in preferred embodiments the second precipitate comprising the second resolving agent and the first enantiomer is in the form of a complex.

In the resolution process of the present invention, it is preferred for said first enantiomer to be (R) 3-hydroxy-3-(2-phenylethyl)-hexanoic acid (hereinafter sometimes referred to for convenience as the “R-acid”) and said second enantiomer to be (S) 3-hydroxy-3-(2-phenylethyl)-hexanoic acid (hereinafter sometimes referred to for convenience as the “S-acid”). It is most preferred if the R-acid and the S-acid are in the form of a racemic mixture. Although it is contemplated that all aprotic, polar solvents may be adaptable for use in accordance with the present invention, the preferred aprotic, polar solvent is acetonitrile, or acetonitrile in combination with one or more other aprotic, polar solvents. In the same manner, it is contemplated that any one of the available non-polar, aprotic solvents may be adaptable for use in accordance with the present invention. In general, however, the preferred aprotic non-polar solvent is toluene or toluene in combination with one or more aprotic, nonpolar solvents. The preferred resolving agent for said first enantiomer is L-(−)-norephedrine and the preferred resolving agent for said second enantiomer is D-(+)-norephedrine. It will be appreciated, however, that other resolving agents may be used.

Another aspect of preferred embodiments of the present invention is the provision of a process for resolving an acetonitrile racemate solution of R-acid and S-acid from an acetonitrile solution of the racemate comprising precipitating the S-acid from the solution followed by separation of the precipitate from the solution, preparing the solution for a second precipitation, then precipitating the R-acid from the solution, and separating the precipitated R-acid substantially free of residual solution. It is preferred to precipitate the S-acid and R-acid with conventional resolving agents. While not wanting to be bound by theory, when conventional resolving agents are employed it is believed that the S-acid and R-acid precipitates are in the form of neutralization complexes comprising, respectively, a first resolving agent and the S enantiomer of the hydroxy acid (S-acid) and a second resolving agent and the (R) enantiomer of the hydroxy acid (R-acid).

Another aspect of preferred embodiments of the present invention is the provision of a process for synthesizing [R—(R*,R*)]-N-[3-[1-[5,6-dihydro-4-hydroxy-2-oxo-6-(2-phenylethyl)-6-propyl-2H-pyran-3-yl]propyl]phenyl]-5-(trifluoromethyl)-2-pyridinesulfonamide (the target antiviral compound) comprising the steps of:

(A) providing 1-phenyl-hexan-3-one;

(B) providing a racemic mixture of 3-hydroxy-3-(2-phenylethyl)-hexanoic acid by reacting the 1-phenyl-hexan-3-one provided in Step (A) with ethylbromoacetate under Reformatsky conditions to produce 3-hydroxy-3-(2-phenylethyl)-hexanoate ethyl acetate and sapponifying the acteate with aqueous sodium hydroxide;

(C) isolating (R)-3-hydroxy-3-(2-phenylethyl)-hexanoic acid (R-acid) in enantiomeric excess by saponification and reverse resolution of the racemate of step (B) to produce a resolved product;

(D) acidifying the resolved product of step (C) to produce the free acid;

(E) synthesizing a keto-ester comprising (R)-ethyl-5-hydroxy-3-oxo-5-phenyethyl-octanoate by reaction of said (R)-3-hydroxy-3-(2-phenylethyl)-hexanoic acid from step (D) with monoethyl malonate magnesium salt in the presence of 1,1′-carbonyldiimidazole;

(F) converting the keto-ester of step (E) to a hydroxy-lactone intermediate comprising (6R)-5,6-dihydro-4-hydroxy-6-[1-(2-phenylethyl)]-6-propyl-2H-pyran-2-one;

(G) converting the hydroxy-lactone intermediate of step (F) to the nitro-propenyl compound comprising [3α(R),6(R)]-5,6-dihydro-4-hydroxy-3-[(Z)-1-(3-nitrophenyl)-propenyl]-6-[1-(2-phenylethyl)]-6-propyl-2H-pyran-2-one and its enantiomer by condensing the hydroxy-lactone intermediate with m-nitropropiophenone in the presence of titanium tetrachloride and pyridine;

(H) stereospecifically reducing said nitro-propenyl compound from step (G) to the nitro-propyl compound comprising [3α(R),6(R)]-5,6-dihydro-4-hydroxy-3-[1-(3-nitrophenyl)-propyl]-6-[1-(2-phenylethyl)]-6-propyl-2H-pyran-2-one by contact with [(1,5-cyclooctadiene)rhodium(I)-1,2-bis-(2R,5R)-dimethyl-phospholano)benzene]tetrafluoroborate in the presence of hydrogen;

(I) reducing the nitro-propyl compound of step (H) to the amino-propyl compound comprising [3α(R),6(R)]3-[1-(3-aminophenyl)-propyl]-5,6-dihydro-4-hydroxy-6-[1-(2-phenylethyl)]-6-propyl-2H-pyran-2-one by contact with a palladium catalyst in the presence of hydrogen; and

(J) reacting the amino propyl compound of step (I) with 5-(trifluoromethyl)-2-pyridinesulfonyl chloride in the presence of pyridine followed by acidification of the mixture, thus providing [R—(R*,R*)]-N-[3-[1-[5,6-dihydro-4-hydroxy-2-oxo-6-(2-phenylethyl)-6-propyl-2H-pyran-3-yl]propyl]phenyl]-5-(trifluoromethyl)-2-pyridinesulfonamide.

Preferably, step (A) is carried out by hydrogenation of the product of a condensation reaction between 2-pentanone and benzaldehyde.

### DETAILED DESCRIPTION OF THE PREFERRED EMBODIMENTS

In a process for the synthesis of the antiviral compound described above, applicants have discovered methods to improve the prior art synthetic scheme. As described above, in the prior art process for the synthesis of the antiviral compound, the net yield of product from the two steps involving the synthesis and isolation in high optical purity of (R) 3-hydroxy-3-(2-phenylethyl)-hexanoic acid is relatively low. In a preferred embodiment of the present invention, the synthesis of the antiviral compound, outlined in reaction Scheme II below, comprises an improved process for synthesizing 3-hydroxy-3-(2-phenylethyl)-hexanoic acid (the hydroxy acid), and a new process for separating, and preferably isolating, the (R) enantiomer of the hydroxy acid (R-acid) therefrom. The R-acid is a critical intermediate in the synthesis of the antiviral compound according to certain synthesis schemes, described in detail above.

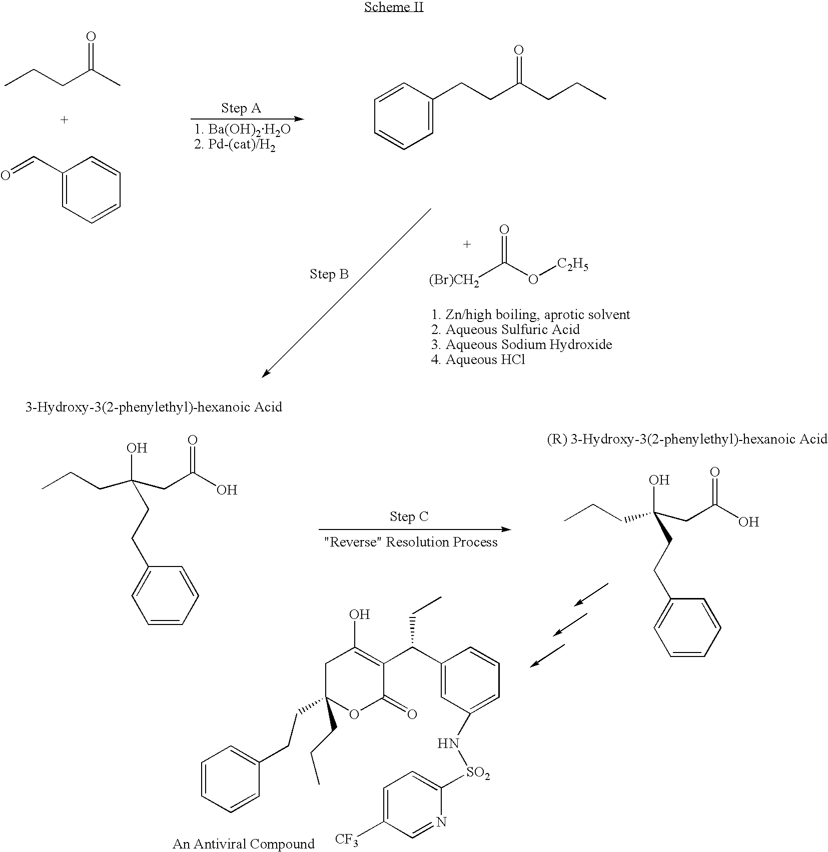

With reference to reaction Scheme II, applicants' improved process for the synthesis of the antiviral compound preferably begins with the synthesis of 1-phenyl-hexan-3-one in step (A), preferably utilizing a base catalyzed reaction, to condense 2-pentanone and benzaldehyde, followed by treatment, preferably in situ, of the reaction mixture with catalyst under reducing conditions, preferably palladium metal catalyst in the presence of hydrogen, and reducing the products of the condensation to yield 1-phenyl-hexan-3-one (the ketone).

In preferred embodiments, the ketone produced in step (A) is separated, and preferably isolated, and used in the Reformatsky reaction of step (B) to produce the hydroxy acid. In the Reformatsky reaction of step (B), the ketone is preferably reacted with ethyl-bromoacetate under Reformatsky conditions (that is, in the presence of zinc metal and optionally an activator) to produce the hydroxy acid. It is highly preferred that step (B) is carried out in an aprotic, high boiling reaction solvent, for example, dimethoxy ethane (DME), dimethoxy methane (DMM), or toluene. It is preferred to use mixtures of aprotic solvents, for example mixtures of DME and toluene or DMM and toluene. Particularly preferred are mixtures of aprotic solvents compromising at least about 25 vol % DME and up to about 75 vol % toluene.

In step (B), when the condensation reaction has run to a substantial extent, preferably substantially to completion, the product, ethyl-3-hydroxy-3-(2-phenylethyl)hexanoate (an ester of the hydroxy acid, hereinafter, the hydroxy ester), is preferably liberated by workup of the reaction mixture with aqueous sulfuric acid. After neutralization and washing, the reaction mixture is next contacted with, and preferably intimately mixed with, an aqueous sodium hydroxide solution to saponify the hydroxy ester. Preferably the acid salt is concomitantly extracted into the aqueous solution. The hydroxide/hydroxy acid solution is then acidified, and the free 3-hydroxy-3-(2-phenylethyl)hexanoic acid (the hydroxy acid) is extracted from the acidified aqueous solution, preferably into toluene. Details of the preferred aspects of step (B) of the improved synthesis are further described in the examples below.

In the preferred step (C) of Scheme II, the free hydroxy acid, which is preferably extracted into toluene, is separated (resolved) using the reverse resolution process of the present invention, described in detail below. The separated R-acid is converted to the antiviral compound according to the remainder of the synthesis scheme described above in Scheme I or any other scheme which is now known or becomes known to those skilled in the art. It will be appreciated that either pathway described in Scheme I can be used to provide the antiviral compound, but in the improved synthetic process of the present development, Pathway (A) of Scheme I is preferred.

Next, preferred embodiments of applicants' improvements will be described in greater detail. First will be described preferred aspects of the process improvement step comprising the Reformatsky synthesis of racemic 3-hydroxy-3-(2-phenylethyl)-hexanoic acid, followed by a detailed description of the preferred reverse resolution process for resolving racemic acids.

### Reformatsky Synthesis of 3-Hydroxy-3-(2-phenylethyl)-hexanoic Acid

It has been recognized that in the synthesis of the antiviral compound described above, the critical intermediate (R)-3-hydroxy-3-(2-phenylethyl)-hexanoic acid (the R-acid) is a fragile compound which is easily decomposed upon heating. In point of this fact, art-recognized synthesis of the hydroxy acid has heretofore been carried out at temperatures less than about −58° C.

Reformatsky reactions are the condensation of an aldehyde or ketone with a halo-substituted ester, and are generally carried out at room temperature or higher. As is known, typically the products sought from a Reformatsky reaction are the unsaturated enol derivative products of a hydroxy ester. In addition, Reformatsky reactions are known to exhibit side reactions comprising self-condensation of the carbonyl compound used in the reaction as well as enolization of the product, yielding an unsaturated ester. Often, work-up of these product mixtures to obtain a hydroxy acid in pure form is difficult, and yields may not be high. These chracteristics of Reformatsky reactions are further described in Organic Reactions, Rathke, 1975, vol 22, pp 423-458.

However, applicants have come to appreciate and understand that, notwithstanding the issues described above, it is possible to develop a highly desirable and advantageous process for use in the synthesis of compounds such as the antiviral compound described above which are based on Reformatsky reactions. The Reformatsky reaction has not been considered heretofore as a synthetic methodology for the synthesis of ethyl-3-hydroxy-3-(2-phenylethyl)-hexanoate from the 1-phenyl-hexan-3-one in high yields of easily purified product.

Applicants are the first to recognize the advantage and to observe the superior results achieved with the use of a high boiling reaction solvent to produce hydroxy acid compounds based on the Reformatsky reaction. Examples of preferred solvents that provide a reaction environment facilitating synthesis of the hydroxy acid in high yield and with low yields of side products under Reformatsky conditions are those that comprises at least about 25 vol % up to 100 vol % dimethoxy ethane (DME) or dimethoxy methane (DMM). Toluene is also sufficiently high boiling to provide a reaction solvent in which the Reformatsky synthesis method of the present invention can be carried out. Particularly preferred is a solvent system comprising about 25 vol. % DME or DMM and about 75 vol. % toluene.

Accordingly, applicants have discovered that the prior art Aldol condensation of 1-phenyl-hexan-3-one with ethyl acetate carried out at a temperature between about −50 to about −70° C. in the presence of either lithium diisopropyl amide or butyl lithium can be replaced with a Reformatsky condensation, preferably carried out at a temperature of between about +60 to about +100° C., and even more preferably using reactants of moderate reactivity in the above-described solvent system. As used herein, the term moderate reactivity means that the reactants are substantially stable under ambient environmental conditions.

Thus, substituting applicants' Reformatsky process for the prior art Aldol condensation is a substantial and important improvement over the prior art synthesis of the hydroxy acid with respect to one or more of the following: (a) the rate of the synthesis (in preferred embodiments the reaction is completed in 30% of the time required for low temperature Aldol reaction); (b) the reactivity of the reagents employed (the preferred Reformatsky reaction of the present invention uses less agressive reagents); (c) the temperature regime in which the reaction is run (the need for specialized equipment to achieve sub-ambient conditions is eliminated); and (d) the overall conversion efficency of the starting materials. It has been found that when applied to the synthesis of the critical intermediate hydroxy acid (described above) the Reformatsky condensation step of the present invention can provide yields in excess of 97 mole % conversion based on starting ketone.

These improvements also relate to the provision of a reaction which is more easily scaled-up to a size commensurate with the preparation of commercial quantities of the critical intermediate needed for the synthesis of the antiviral compound, that is, the R-acid. The improvement in scalability is due to the less reactive (ie. more stable at ambient conditions) nature of the reagents used to drive the reaction (activated zinc replaces highly reactive lithium reagents), and the elimination of the need for sub-ambient temperatures during the reaction. The less reactive nature of the reagents used in the reaction permits the use of less specialized handling equipment for measuring and transferring the reagents, and provides for an additional margin of safety during large scale manufacture. The elimination of the requirement for sub-ambient temperature in the condensation reaction permits industrial-scale synthesis to be carried out in common place industrial equipment, eliminating the necessity of high cost, cryogenic equipment required for low temperature reactions.

The details of the preferred conditions required to use a Reformatsky reaction to couple ethyl-haloacetate to 1-phenyl-hexan-3-one in the production of ethyl-[3-hydroxy-3-(2-phenylethyl)]-hexanoate (the hydroxy acid) which, as mentioned above is a critical intermediate in the synthesis of the above-described antiviral compound, are outlined in Scheme III, below:

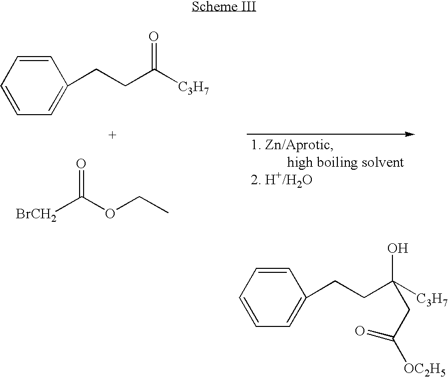

In the preferred Reformatsky reaction process of the present invention, activated zinc metal is contacted with an ethyl-halo-acetate in the presence of 1-phenyl-hexan-3-one yielding ethyl-3-hydroxy-3-(2-phenylethyl)-hexanoate. Preferably the reaction is carried out in an aprotic, high boiling point solvent of moderate polarity. While it is contemplated that a large number of aprotic, high boiling components can be used as the solvent, it is preferred that the solvent comprises, and even more preferably consists essentially of, one or more of dimethoxy ethane (DME) and toluene. The preferred reaction is carried out by introducing cleaned zinc metal into a reaction vessel and then adding an activator, the reaction solvent, and the ketone. The reaction mixture is then brought to reflux and, preferably with mixing, the ethyl-haloacetate-ester is added, preferably in two aliquots. A first aliquot, preferably comprising about 20 wt. % of the total amount of the acetate-ester needed to complete the reaction, is added relatively quickly (eg., over a period of about less than about 2 minutes) thereby initiating the reaction. Then, preferably after the initial reaction has substantially subsided, the remainder of the acetate-ester is added, preferably slowly, (eg., over a period of about 30 minutes) preferably with continued refluxing. Once all of the acetate-ester has been added, the reaction mixture preferably is refluxed for about one additional hour. Thereafter, the mixture is cooled, the solids filtered off and the filtered reaction mixture is contacted, preferably by mixing, with an aqueous sulfuric acid solution, liberating the hydroxy-ester and removing from the reaction mixture any remaining reactive metal complexes.

The reaction mixture is then preferably contacted (eg., by mixing) with an aqueous sodium hydroxide solution to liberate the acid salt from the ester. During contact with the sodium hydroxide solution, the acid salt is extracted into the aqueous solution. The aqueous solution containing the acid salt is then acidified, yielding the free hydroxy acid, and the free hydroxy acid can be extracted from the aqueous solution by mixing with a non-protic solvent of low polarity, for example, toluene. Once extracted into an organic solvent, the free hydroxy acid can be isolated by removing the organic solvent, for example, by vacuum distillation.

The reagents and procedure for carrying out a Reformatsky coupling reaction have been described in detail by Rathke in Organic Reactions, 22, 1975, pp 423-458, the description of which is incorporated herein by reference. In the present invention process, reduction of unwanted side reactions has been accomplished by using a high boiling reaction solvent. The preferred solvent preferably comprises at least about 25 vol %, preferably from about 25 vol % to about to about 100 vol %, of a medium polarity, aprotic, high boiling solvent. While a large number of components are contemplated for use in this regard, and all such components are within the scope of the present invention, preferred solvents include dimethoxy ethane and dimethoxy methane. Optionally, the preferred reaction solvent can comprise about 75 vol % or less of another aprotic solvent, for example toluene. While the best results are observed in reaction solvents consisting of essentially of DME or mixtures of DME and toluene, good results, in terms of rapid reaction times and low occurrence of side products are also seen in a reaction solvent consisting essentially of toluene.

In the improved process of the present invention, any of the forms of zinc metal which are known to be active in Reformatsky reactions can be employed, for example, zinc granulate, 20 mesh zinc metal, or zinc powder. It will be appreciated that any of the procedures generally known to be effective for preparation of the surface of the zinc for use in a Reformatsky reaction can be employed. Examples of such surface preparation procedures include washing the metal with acid followed by a rinse with deionized water and baking dry.

It will be appreciated that any of the activators known to promote the Reformatsky reaction can be used in the hydroxy acid synthesis of the present invention, examples of which include iodine and trimethylsilyl-chloride. It will be appreciated that generally, only a nominal wt. % of the activator relative to the amount of metal used needs to be added to the reaction mixture, but various conditions of the reaction, for example, the source or purity of the zinc used, may dictate more or less activator be employed as will be familiar to one of ordinary skill in the art.

It will be appreciated that the prior art process for the synthesis of the antiviral compound may be improved solely by substitution of the prior art Aldol condensation step with a Reformatsky condensation step according to applicants' description, and that further improvement may be additionally realized, as described below, by also substituting the racemate resolution step of the prior art process with the reverse resolution process of the present invention, the preferred embodiments of which are described next.

### Reverse Resolution of Racemic 3-Hydroxy-3-(2-phenylethyl)-hexanoic Acid

With reference to Scheme II, the reverse resolution process of Step (C) is based on Applicants' recognition that a source of the problem of low recovery and low optical purity in the prior art processes for resolving hydroxy acid racemates is related, at least in part to the relative concentration of the desired and unwanted enantiomers in the solution from which the desired enantiomer is precipitated. Applicants believe that they are the first to recognize that if the concentration of the unwanted enantiomer is reduced in a solution containing two enantiomers of a hydroxy acid, a correspondingly greater proportion of the desired enantiomer in solution can subsequently be precipitated and recovered by using an art-recognized resolving agent. Thus applicants' “reverse resolution” process comprises first reducing the amount of unwanted enantiomer present in a racemate solution by precipitation of the unwanted enantiomer, followed by (after separation of the first precipitate and the supernatant solution) precipitation and recovery of the desired enantiomer from the solution. As will be apparent from the description of the process, below, applicants' reverse resolution process provides improvement of both the percentage of the desired acid enantiomer recovered from the racemic mixture and of the optical purity of the recovered product.

As applied to the preferred resolution of racemic 3-hydroxy-3-(2-phenylethyl)-hexanoic acid and separation of optically pure (R) 3-hydroxy-3-(2-phenylethyl)-hexanoic acid therefrom, the reverse resolution process comprises the following steps:

a) providing a first mother liquor comprising a mixture of (R) and (S) 3-hydroxy-3-(2-phenylethyl)-hexanoic acid in acid form dissolved in acetonitrile;

b) contacting the first mother liquor with (D)(+)-norephedrine;

c) forming a first precipitate comprising a complex of (S) 3-hydroxy-3-(2-phenylethyl)-hexanoic acid and (D)(+)-norephedrine;

d) separating, said first mother liquor substantially free from said first precipitate;

e) forming a second mother liquor by substantially replacing said acetonitrile in said first mother liquor with toluene;

f) contacting said second mother liquor with aqueous HCl;

g) subsequent to step (f), forming a third mother liquor by substantially replacing said toluene in said second mother liquor with acetonitrile;

h) contacting said third mother liquor with with (L)(−)-norephedrine;

i) forming a second precipitate comprising a complex of (R) 3-hydroxy-3-(2-phenylethyl)-hexanoic acid and (L)(−)-norephedrine; and

j) isolating said second precipitate substantially free of said third mother liquor.

Thus in the preferred embodiments of the present resolution process, in a first precipitation step the racemate solution is treated with the “wrong” chiral resolving agent to precipitate as much of the “unwanted” hydroxy acid enantiomer as may be precipitated without unacceptable loss of the “desired” hydroxy acid enantiomer. Preferrably, at least about 20% of the “unwanted” enantiomer (the S-acid in the illustrative process) will be removed, more preferably at least about 70%, and most preferable at least about 90% of the unwanted enantiomer will be removed by precipitation. It is preferred for most of the desired enantiomer (the R-acid in the illustrative process) to remain in solution after the first precipitation, preferably at least about 90% and more preferably at least 98% of the desired (R-acid) enantiomer to remain in solution after the first precipitation. In the present invention process, the material precipitated in the first precipitation step is separated from the solution containing the “desired” enantiomer (first mother liquor). This separation is followed by a second precipitation step in which the “correct” resolving agent is used to precipitate the desired enantiomer of hydroxy acid from the solution.

In precipitating an amount of the “unwanted” enantiomer from the racemate solution in the first step, the concentration of the “unwanted” enantiomer is reduced, and the amount of the “desired” enantiomer which can be precipitated in the second step is thereby increased. Between precipitation steps (c) and (i), the solution containing the desired enantiomer is acidified by contact with aqueous acid, preferably by intimate mixing (step f), which preferably ensures that all of the hydroxy acid in solution is in an acid form. This step also preferably extracts any of the resolving agent remaining in solution which complexes with the “unwanted” enantiomer, reducing or eliminating co-precipitation of unwanted enantiomer with the desired enantiomer in the second precipitation step. To facilitate the acidification and extraction step, the polar solvent from which the “wrong” enantiomer of hydroxy acid was precipitated in step (c) is exchanged for a non-polar solvent, for example, toluene, in step (e) prior to acidification of the mother liquor containing the desired enantiomer. Following acidification, the non-polar solvent is exchanged for a polar solvent in step (g) to facilitate the second precipitation step.

Using the above-described reverse resolution process to resolve 3-hydroxy-3-(2-phenylethyl)-hexanoic acid, the R-acid can be separated from a racemate of the hydroxy acid to yield a product having a chemical purity in excess of 99 mole %, optical purity in excess of 99% enantiomeric excess (ee), and yields of up to 98 mole % of the R-acid initially present in the racemic mixture. These results represent considerable improvement over the prior art, for example, the above-mention results reported by Fors et al. in the Journal of Organic Chemistry, Vol. 63 (1998), pp. 7348-56, which reports yields the R-acid at a chemical purity of 98 mole % and 98% ee, albeit at an overall yield (isolation yield) of about 50 mole % based on the amount of the (R) isomer of the hydroxy acid present in the racemic mixture. Thus, improvement of both overall yield and optical purity of separated hydroxy acid is accomplished by the process of applicants' invention which utilizes two precipitation steps of the specific nature described herein instead of a single precipitation step used in art-recognized resolution processes.

With reference to Scheme I, it will be appreciated that the above-identified prior art process for the synthesis of the antiviral compound may be improved solely by substitution of the art-recognized resolution step with the reverse resolution process of the present invention. It will also be appreciated that further improvement may be realized, as described above, by additionally substituting the Aldol condensation step of the prior art process with the aforementioned Reformatsky condensation reaction.

It will be appreciated that the resolution process of the present invention may be applied to many different hydroxy acids as well as other chiral compounds having only one enantiomer which forms a substantially insoluble complex with a particular resolving agent. Thus, the following steps comprise a preferred form of the present resolution process for a mixture comprising an organic acid first enantiomer (the isolation of which in an optically pure form is desired) and second enantiomer (the presence of which is generally unwanted in and/or detrimental to the purified compound), the process comprising:

(a) providing a first mother liquor comprising a mixture of a first and a second optically active enantiomer in acid form dissolved in a polar, aprotic solvent;

(b) contacting the first mother liquor with a first resolving agent known to form with said second enantiomer a precipitate which is not more than sparingly soluble in said first mother liquor, said resolving agent further characterized in that it does not form the first enantiomer any substantial amount of precipitate from said first mother liquor;

(c) forming a first precipitate comprising said first resolving agent and said second enantiomer;

(d) separating said first mother liquor substantially free from said first precipitate;

(e) subsequent to step (d), forming a second mother liquor by replacing said polar, aprotic solvent in said first mother liquor with a non-polar, aprotic solvent;

(f) contacting said second mother liquor with an aqueous acid solution;

(g) forming a third mother liquor by replacing said non-polar, aprotic solvent of said second mother liquor with a polar, aprotic solvent;

(h) contacting said third mother liquor with a second resolving agent capable of forming with said first enantiomer a precipitate which is not more than sparingly soluble in said third mother liquor;

(i) forming a second precipitate comprising said first enantiomer and said second resolving agent; and

(j) isolating said second precipitate substantially free of said third mother liquor

It is to be understood that reference to a “sparingly soluble complex” includes a complex which is sparingly soluble under the solution conditions which obtain upon contact by the resolving agent as well as a complex which can be rendered sparingly soluble by alteration of the solution conditions after contact by the resolving agent, for example, by cooling the solution to precipitate a complex after contacting the solution with a resolving agent.

It will be appreciated also that the inventive resolving process can in general be applied to separating the enantiomers of any optically active active organic compound from a mixture comprising a first enantiomer, the separation of which is desired, and a second enantiomer, the presence of which is unwanted, the process comprising:

(a) providing a first solution comprising: the first enantiomer; the second enantiomer; and a solvent;

(b) contacting said first solution with a first resolving agent under conditions effective to precipitate from said first solution at least a portion of said second enantiomer, thereby forming a supernatant solution comprising the solvent and at least a portion of said first enantiomer;

(c) preparing said supernatant solution from step (b) for a second precipitation characterized in that said prepared supernatant solution does not contain any substantial amount of said first resolving agent and a substantial portion of said first enantiomer in the supernatant solution is in a form suitable to form a precipitate with said second resolving agent; and

(d) subsequent to step (c), contacting said prepared supernatant solution with a second resolving agent under conditions effective to precipitate at least a portion of said first enantiomer contained in said supernatant solution, said precipitate being characterized in that it does not contain any substantial amount of said second enantiomer.

In preferred embodiments, the preparing step (c) comprises:

(i) separating the supernatant solution from the precipitate;

(ii) optionally, replacing at least a portion of the solvent in the separated supernatant solution with at least one solvent of different polarity;

(iii) subsequent to step (ii), contacting said supernatant solution with a sufficient amount of an immiscible liquid phase under conditions sufficient to cause a substantial portion of any of said first resolving agent remaining in said supernatant solution to migrate from said supernatant solution to said immiscible liquid phase and wherein said contact places at least some, preferably substantially all, of said first enantiomer in a condition suitable for forming a precipitate with a second resolving agent;

(iv) separating said supernatant solution from said immiscible liquid phase; and

(v) subsequent to step (iv), if step (ii) was carried out, replacing at least a portion of the supernatant solution comprising the solvent of different polarity introduced in step (ii) with the same solvent comprising the supernatant solution in step (i) or a solvent having the same or substantially similar polarity as the solvent comprising the supernatant solution of step (i).

It is to be understood that reference to “conditions efffective to participate at least a portion of the complex” refer to any changes which will yield a useful separation of the complex from the solution. It is also to be understood that reference to a composition which does not contain any “substantial amount” of an identified substance refers to a composition in which the identified substance is not present in an amount which precludes the usefulness of the separation process.

### EXAMPLES

There follows a description of the preparation of the hydroxy acid using Reformatsky conditions according to the present invention process, as described above, followed by a description of the use of the high-yield, high-purity resolution process of the present invention to isolate the “R” enantiomer of the hydroxy acid from the racemate produced in the example Reformatsky reaction.

Unless otherwise noted, all reagents were obtained from Aldrich. Commercial grade solvents were used without additional drying or purification by distillation. All reactions were run under a nitrogen blanket. All reagents were used as received unless otherwise noted.

### Example 1

### Preparation of Ethyl-3-hydroxy-3-(2-phenylethyl)hexanoic Acid

Into a reaction vessel fitted with a mechanical stirrer, a heating bath, and a reflux condenser was placed 37.5 ml of toluene and 12.5 ml of dimethoxy ethane (DME). About 10 g of 1-phenyl-3-hexanone, 5.6 g of zinc dust (ECKA WEKK, Germany >99.9%, 0.045 mm mesh size) and about 10 mg of iodine (Riedel-deHaen, 99.8%) were added to the reaction vessel with stirring. The vessel was sealed and the contents heated to reflux. The vessel was refluxed for one hour, following which 1 g of ethyl bromoacetate (Aldrich, 98%) was added by dropping funnel all at once. During the addition of the bromoacetate evidence of an exotherm (foaming of the reaction mixture) was observed. After the initial exotherm, and additional 11.3 g of ethyl-bromo-acetate was added dropwise to the reaction vessel over about thirty minutes.

After the addition of ethyl-bromo-acetate, refluxing of the reaction mixture was continued for about one hour additional. Following this period of reflux, the reaction mixture was cooled to about 50° C., and the insoluble materials separated from the toluene/DME solution (organic phase) by vacuum filtration. The solid materials were extracted with 10 ml of a solvent comprising about 75 vol % toluene and about 25 vol % DME. The extract was added to the organic phase.

Thus isolated, the organic phase was stirred with 65 g of 12 wt. % aqueous sulfuric acid for about thirty minutes. The organic phase was separated from the aqueous phase and washed with water followed by a 10 wt. % sodium sulfate aqueous solution.

The organic layer was separated from the wash solution and transferred to a second reaction vessel. To the reaction solution was added about 22 ml of methanol (reagent grade, Aldrich). The solution was then cooled to about 20° C. on an ice bath and 44 ml of 2M aqueous sodium hydroxide was added. The mixture was stirred for 24 hours.

At the end of 24 hours, the phases were seperated and the organic phase discarded. The retained aqueous phase was cooled to about 10° C. and 12.6 ml of 25 wt. % aqueous HCl was added to it. The acidified aqueous layer was extracted with about 44 ml of toluene. The toluene was separated from the aqueous layer and the toluene was removed by vacuum distillation at about 15 torr to leave a yellow oil comprising more than about 90% racemic hydroxy acid and less than about 10% toluene by comparison of GC peak areas, according to published procedures. The oil prepared in this manner was used without further purification in the resolution process of the present invention.

### Example 2

### Large Scale Preparation of Ethyl-3-hydroxy-3-(2-phenylethyl)hexanoic Acid

Preparation of ethyl-3-hydroxy-3-(2-phenylethyl)hexanoic acid (the hydroxy acid) was carried out on a commercial scale to demonstrate the applicability of the preparative scheme of the present invention to commercial preparation of the critical intermediate hexanoic acid compounds. Thus, the acid precursor was prepared on the kilogram scale using a 100 liter glass lined reaction vessel fitted with a mechanical stirrer and a reflux condensor. The reactor also was equipped with a oil heat jacket. All reagents used in the scale-up preparation were technical grade, articles of commerce, used as received.

Into the reactor was placed 20.6 kg of toluene, 8.7 kg of dimethoxyethane (DME), 3.52 kg of zinc dust, 7.8 kg of 1-phenyl-3-hexanone and about 7.0 g of iodine crystals (a catalytic amount). The reactor was sealed and purged with nitrogen. The reaction mixture was stirred and heated to reflux (about 75° C.). Refluxing was continued at this temperature for one hour.

After one hour, the reaction mixture was heated to 85° C. and over the next 90 minutes, 9.25 kg of ethyl-bromo-acetate was added. It was observed that reaction occurred immediately upon addition of the acetate with no initiation period.

Upon completion of ethyl-bromoacetate addition, the temperature of the reaction mixture was reduced to 65° C. and held at that temperature with continued stirring for one hour. At the end of one hour, the reaction mixture was cooled to 50° C. and the solids were removed by filtration. The filter cake was washed with a 2.5:1 w/w mixture of toluene:DME (approximately 10 L in a single aliquot) and the wash was added to the filtrate.

The filtrate was added to 46.25 kg of 13.5 wt % aqueous sulfuric acid which had been cooled to less than 5° C. The mixture was stirred for about 3 hours at ambient temperature then left to stand to effect separation of the organic and aqueous phases. The organic phase was decanted from the aqueous phase and washed with a 10 wt % aqueous sodium sulfate solution. A sample of the organic phase was subjected to GC analysis, which showed that 96.5 mole % of the starting hexanone was converted to the hydroxy acid.

### Example 3

### Isolation of (R)-3-Hydroxy-3-(2-phenylethyl)-hexanoic Acid

About 14.2 g of the racemic (RS)-3-hydroxy-3-(2-phenylethyl)-hexanoic acid (the hydroxy acid) prepared in Example 1, above, was placed into a vessel and dissolved in 90 ml of acetonitrile. Into this was dissolved 3.9 g of (+)-norephedrine added as one aliquot, with stirring. The solution was stirred at ambient temperature (about 20-25° C.) for about 16 hours, then cooled to about 0-5° C. and stirred for about three hours additional. Over the course of stirring, a white precipitate was formed. The white precipitate was separated from the solution by vacuum filtration. The solids thus obtained were washed with 25 ml of 0-5° C. acetonitrile. The wash was added to the filtrate, and the volatiles were removed by vacuum distillation at about 15 torr.

The residue isolated from the filtrate was redissolved in about 40 ml of toluene. The toluene solution was extracted with about 40 ml of 25 wt. % aqueous HCl, and separated from the aqueous layer. Toluene was vacuum distilled from the isolated organic layer, leaving an oily residue which was redissolved in about 90 ml of fresh acetonitrile at ambient temperature (about 20-25° C.).

Into the ambient temperature acetonitrile solution was added, all at once, about 3.9 g of (−)-norephedrine, with stirring, forming a suspension. The suspension was stirred under ambient conditions for about 16 hours, then cooled to a temperature of about 0-5° C. and stirred for an additional three hours. During stirring a precipitate was formed. The precipitated material was isolated from the acetonitrile solution at sub-ambient temperature by vacuum filtration. The filter cake was washed with about 25 ml of acetonitrile at a temperature of about 0-5° C.

The solids in the filter cake were recrystallized by slurrying them in about 90 ml of acetonitrile contained in a vessel fitted with a reflux condenser and heating the slurry to reflux conditions until a clear solution was obtained. The solution was then gradually cooled to precipitate the crystalline salt of the (R) form of the hydroxy acid. The crystalline material was isolated by vacuum filtration and the recrystallization process described above was repeated, and the crystalline material dried under vacuum of about 15 torr while maintaining the temperature of the material at less than about 40° C.

The purity of the isolated hydroxy acid was assayed by published HPLC methods and found to be 99.8 mole % hydroxy acid. Of the hydroxy acid isolated, the optical purity was found to be 99.9 mole % pure (i.e., about 0.05 mole % of the S-acid was present in the isolated product).

HPLC assay indicates that the resolution process of the present invention yielded about 98 mole % recovery of the R-acid that was present in the starting racemic mixture.

With reference to Scheme I, as described above, it will be appreciated that a hydroxy-lactone intermediate compound to the antiviral compound described above can be prepared from the (R) enantiomer of the hydroxy acid which was prepared according to Examples 1 or 2 and/or isolated according to Example 3, above, utilizing the synthetic scheme detailed in published international application WO 99/12919.

What is claimed is:

1. A process for separating a first enantiomer of an optically active organic compound from a mixture comprising a first enantiomer and a second enantiomer, the process comprising:
(a) contacting a first solution comprising: (i) the first enantiomer; (ii) the second enantiomer; and (iii) a solvent, with a first resolving agent under conditions effective to precipitate from said first solution at least a portion of said second enantiomer, thereby forming a supernatant solution comprising at least a portion of said solvent and at least a portion of said first enantiomer;
(b) preparing said supernatant solution from step (a) for a second precipitation characterized in that: (i) said prepared supernatant solution does not contain any substantial amount of said first resolving agent; and (ii) a substantial portion of said first enantiomer in the supernatant solution is in a form suitable to form a precipitate with a second resolving agent; and
(c) subsequent to step (b), contacting said prepared supernatant solution with a second resolving agent under conditions effective to precipitate at least a portion of said first enantiomer contained in said solution, said precipitate being characterized in that it does not contain any substantial amount of said second enantiomer.

2. The process of claim 1, wherein said preparing step (b) comprises:
(i) separating said supernatant solution from the precipitate;
(ii) optionally, replacing at least a portion of the solvent in said separated supernatant solution with at least one solvent of different polarity;
(iii) subsequent to step (ii) when step (ii) is present, contacting said supernatant solution with a sufficient amount of an immiscible liquid phase under conditions sufficient to cause a substantial portion of said first resolving agent remaining in the supernatant solution to migrate from the supernatant solution to said immiscible liquid phase, and whereby said contact places at least some of said first enantiomer in a condition suitable for forming a precipitate with a second resolving agent;
(iv) separating said supernatant solution from said immiscible liquid phase; and
(v) subsequent to step (iv) when step (ii) is present, optionally replacing at least a portion of the supernatant solution comprising the solvent of different polarity introduced in step (ii) with the same solvent comprising the supernatant solution in step (i) or a solvent having the same or substantially similar polarity as the solvent comprising the supernatant solution of step (i).

3. The process of claim 1 wherein said first enantiomer in said steps (a) and (b) and said second enantiomer in step (a) are in acid form, and said solvent in step (a) is a polar, aprotic solvent.

4. The process of claim 3, wherein said preparing step (b) is the process comprising:
(i) separating the supernatant solution from the precipitate;
(ii) replacing at least a portion of the polar, aprotic solvent in the separated supernatant solution with an aprotic, non-polar solvent to produce a substantially non-aqueous, non-polar composition comprising said first enantiomer and said first resolving agent;
(iii) contacting said non-aqueous, non-polar composition with a sufficient amount of an aqueous acid solution under conditions sufficient to cause at least a substantial portion of said first resolving agent to migrate from said non-aqueous, non-polar composition to said aqueous acid solution and to convert substantially all of said first enantiomer in said non-aqueous, non-polar composition to an acid form;
(iv) separating the non-aqueous, non-polar composition containing said first enantiomer in acid form from said aqueous acid phase; and
(v) replacing at least a portion the aprotic, non-polar, solvent in the separated non-aqueous, non-polar composition with a polar, aprotic solvent to produce said second solution comprising a polar, aprotic solvent and said first enantiomer.

5. The process of claim 4 wherein said supernatant solution of said preparing step (b) is substantially free of said first resolving agent.

6. The process of claim 5 wherein the precipitate formed in said contacting step (c) is substantially free of said second enantiomer.

7. The process of claim 6, further comprising recrystallizing the precipitate formed in contacting step (c).

8. The process of claim 7 further comprising isolating said recrystallized precipitate and converting it to a free acid form.

9. The process of claim 8, wherein said first enantiomer is (R) 3-hydroxy-3-(2-phenylethyl)-hexanoic acid, said second enantiomer is (S) 3-hydroxy-3-(2-phenylethyl)-hexanoic acid, said polar, aprotic solvent is acetonitrile, said non-aqueous, non-polar solvent is toluene, said first resolving agent is D-(+)-norephedrine, and said second resolving agent is L-(−)-norephedrine.

10. A process for isolation of a first enantiomer of an optically active organic acid compound in high optical purity from a composition comprising a first enantiomer and a second enantiomer of the compound, the process comprising:
(a) providing a first mother liquor comprising a first and a second optically active enantiomer in acid form dissolved in a polar, aprotic solvent;
(b) contacting the first mother liquor with a first resolving agent which forms with said second enantiomer a precipitate which is not more than sparingly soluble in said first mother liquor, said resolving agent further characterized in that it does not form with said first enantiomer any substantial amount of a precipitate from said first mother liquor;
(c) forming a first precipitate comprising said first resolving agent and said second enantiomer;
(d) separating from said first mother liquor substantially all of said first precipitate;
(e) subsequent to step (d), forming a second mother liquor by replacing said polar, aprotic solvent in said first mother liquor with a non-polar, aprotic solvent;
(f) contacting said second mother liquor with an aqueous acid solution;
(g) forming a third mother liquor by replacing said non-polar, aprotic solvent of said second mother liquor with a polar, aprotic solvent;
(h) contacting said third mother liquor with a second resolving agent which forms with said first enantiomer a precipitate which is not more than sparingly soluble in said third mother liquor;
(i) forming a second precipitate comprising said first enantiomer and said second resolving agent; and
(j) separating substantially all of said second precipitate from said third mother liquor.

11. The process of claim 10 further comprising recrystallizing the precipitate of step (j) from a polar, aprotic solvent.

12. The process of claim 11 further comprising slurrying said recrystallized product in a non-polar, aprotic solvent and converting it to a free acid form by treatment with aqueous HCl.

13. A process for separation of (R) 3-hydroxy-3-(2-phenylethyl)-hexanoic acid from a mixture comprising (R) 3-hydroxy-3-(2-phenylethyl)-hexanoic acid and (S) 3-hydroxy-3-(2-phenylethyl)-hexanoic acid comprising:
(a) contacting a first mother liquor comprising a mixture of (R) and (S) 3-hydroxy-3-(2-phenylethyl)-hexanoic acid in acid form dissolved in acetonitrile with (D)(+)-norephedrine;
(b) forming a first precipitate comprising (S) 3-hydroxy-3-(2-phenylethyl)-hexanoic acid and (D)(+)-norephedrine;
(c) separating said first mother liquor from substantially all of said first precipitate;
(d) forming a second mother liquor by substantially replacing said acetonitrile in said first mother liquor with toluene;
(e) contacting said second mother liquor with aqueous HCl;
(f) subsequent to step (e), forming a third mother liquor by substantially replacing said toluene in said second mother liquor with acetonitrile;
(g) contacting said third mother liquor with with (L)(−)-norephedrine; and
(h) forming a second precipitate comprising (R) 3-hydroxy-3-(2-phenylethyl)-hexanoic acid and (L)(−)-norephedrine.

14. The process of claim 13 further comprising isolating said second precipitate formed in step (h) substantially free of said third mother liquor.

15. The process of claim 14 further comprising recrystallizing said isolated precipitate.

16. In a process comprising a multi-step synthetic scheme for synthesizing [R-(R*,R*)]-N-[3-[1-[5,6-dihydro-4-hydroxy-2-oxo-6-(2-phenylethyl)-6-propyl-2H-pyran-3-yl]propyl]phenyl]-5-(trifluoromethyl)-2-pyridinesulfonamide from (R) 3-hydroxy-3(2-phenylethyl)-hexanoic acid according to Scheme IV: 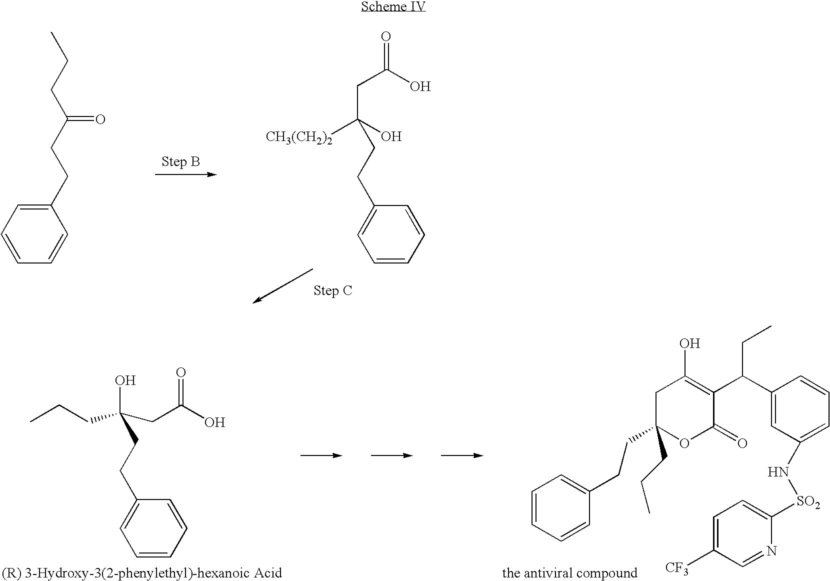
the improvement comprising Step “C” of Scheme IV comprising separation of (R) 3-hydroxy-3-(2-phenylethyl)-hexanoic acid in enantiomeric excess from a mixture of (R) and (S) 3-hydroxy-3-(2-phenylethyl)-hexanoic acid according to the process of claim 15.

17. The process of claim 16 wherein said separation Step C of Scheme IV is performed according to the process of claim 9.

18. The process of claim 16 wherein said separation Step C of Scheme IV is performed according to the process of claim 12.

19. In a process comprising a multi-step reaction sequence for synthesizing [R—(R*,R*)]-N-[3-[1-[5,6-dihydro-4-hydroxy-2-oxo-6-(2-phenylethyl)-6-propyl-2H-pyran-3-yl]propyl]phenyl]-5-(trifluoromethyl)-2-pyridinesulfonamide (the antiviral compound) from (R) 3-hydroxy-3-(2-phenylethyl)-hexanoic acid according to Scheme IV: 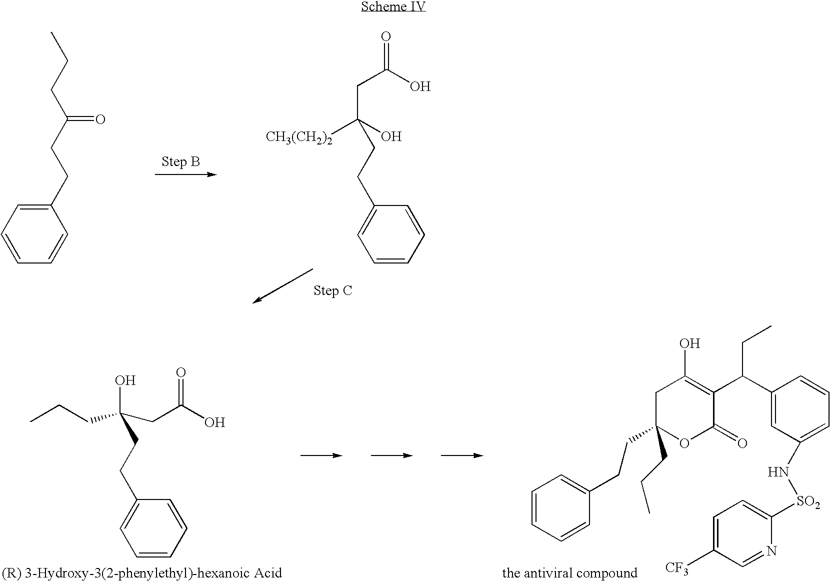
the improvement comprising performing Step “B” of Scheme IV comprising synthesis of racemic 3-hydroxy-3-(2-phenylethyl)-hexanoic acid, by a reaction comprising: contacting 1 phenyl-hexan-3-one with ethyl-bromoacetate in a high boiling, aprotic, polar solvent in the presence of zinc under Reformatsky reaction conditions.

20. The process of claim 19 wherein, during said Step “B” of Scheme IV, said solvent is maintained at a temperature of about 60° C. to about 100° C.

21. The process of claim 20 wherein said solvent used in said Step “B” of Scheme IV comprises at least about 25 vol. % of a solvent selected from the group consisting of dimethoxymethane, dimethoxyethane, toluene, and combinations of two or more of these.

22. The process of claim 21 wherein said Step “C” of Scheme IV comprises separation of (R)3-hydroxy-3-(2-phenylethyl)-hexanoic acid in accordance with the process of claim 9.

23. The process of claim 21 wherein said Step “C” of Scheme IV comprises separation of (R)3-hydroxy-3-(2-phenylethyl)-hexanoic acid in accordance with the process of claim 15.

24. The process of claim 16 further comprising Step “B” of Scheme IV comprising synthesis of 3-hydroxy-3-(2-phenylethyl)-hexanoic acid racemate according to the process of claim 19.

25. The process of claim 16 further comprising Step “B” of Scheme IV comprising synthesis of 3-hydroxy-3-(2-phenylethyl)-hexanoic acid racemate according to the process of claim 20.

26. The process improvement of claim 16 further comprising Step “B” of Scheme IV comprising synthesis of 3-hydroxy-3-(2-phenylethyl)-hexanoic acid racemate according to the process of claim 23.

27. A process for synthesizing [R—(R*,R*)]-N-[3-[1-[5,6-dihydro-4-hydroxy-2-oxo-6-(2-phenylethyl)-6-propyl-2H-pyran-3-yl]propyl]phenyl]-5-(trifluoromethyl)-2-pyridinesulfonamide comprising the steps of:
(a) providing a racemic mixture of 3-hydroxy-3-(2-phenylethyl)-hexanoate ethyl acetate by reacting 1-phenyl-hexan-3-one with ethylbromoacetate under Reformatsky conditions;
(b) isolating (R)-3-hydroxy-3-(2-phenylethyl)-hexanoic acid in enantiomeric excess by saponification and reverse resolution of the racemate of step (a) to produce a resolved product;
(c) isolating the free acid from the resolved product of step (b);
(d) synthesizing a keto-ester comprising (R)-ethyl-5-hydroxy-3-oxo-5-phenyethyl-octanoate by reaction of said (R)-3-hydroxy-3-(2-phenylethyl)-hexanoic acid from step (c) with monoethyl malonate magnesium salt in the presence of 1,1′-carbonyldiimidazole;
(e) converting the keto-ester of step (d) to a hydroxy-lactone intermediate comprising (6R)-5,6-dihydro-4-hydroxy-6-[1-(2-phenylethyl)]-6-propyl-2H-pyran-2-one;
(f) converting the hydroxy-lactone intermediate of step (e) to the nitro-propenyl compound comprising [3α(R),6(R)]-5,6-dihydro-4-hydroxy-3-[(Z)-1-(3-nitrophenyl)-propenyl]-6-[1-(2-phenylethyl)]-6-propyl-2H-pyran-2-one and its enantiomer by condensing the hydroxy-lactone intermediate with m-nitropropiophenone in the presence of titanium tetrachloride and pyridine;
(g) stereospecifically reducing said nitro-propenyl compound from step (f) to the nitro-propyl compound comprising [3α(R),6(R)]-5,6-dihydro-4-hydroxy-3-[1-(3-nitrophenyl)-propyl]-6-[1-(2-phenylethyl)]-6-propyl-2H-pyran-2-one by contact with [(1,5-cyclooctadiene)rhodium(I)-1,2-bis-(2R,5R)-dimethyl-phospholano)benzene]tetrafluoroborate in the presence of hydrogen;
(h) reducing the nitro-propyl compound of step (f) to the amino-propyl compound comprising [3α(R),6(R)]3-[1-(3-aminophenyl)-propyl]-5,6-dihydro-4-hydroxy-6-[1-(2-phenylethyl)]-6-propyl-2H-pyran-2-one by contact with a palladium catalyst in the presence of hydrogen; and
(i) reacting the amino propyl compound of step (g) with 5-(trifluoromethyl)-2-pyridinesulfonyl chloride in the presence of pyridine followed by acidification of the mixture, thus providing [R—(R*,R*)]-N-[3-[1-[5,6-dihydro-4-hydroxy-2-oxo-6-(2-phenylethyl)-6-propyl-2H-pyran-3-yl]propyl]phenyl]-5-(trifluoromethyl)-2-pyridinesulfonamide.
